(19) United States
(12) Patent Application Publication
(10) Pub. No.: US 2004/0260286 A1
(43) Pub. Date: Dec. 23, 2004

(54) Intradiscal devices with anti-extrusion keels

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/894,093

(22) Filed: Jul. 19, 2004

Related U.S. Application Data
(63) Continuation-in-part of application No. 10/007,477, filed on Nov. 8, 2001.
Application No. 10/007,477 is a continuation-in-part of application No. 09/483,805, filed on Jan. 15, 2000, now Pat. No. 6,432,107.
(63) Continuation-in-part of application No. 10/652,842, filed on Aug. 29, 2003.
Application No. 10/652,842 is a continuation-in-part of application No. 10/303,385, filed on Nov. 25, 2002.
Application No. 10/303,385 is a continuation-in-part of application No. 10/191,639, filed on Jul. 9, 2002.
Application No. 10/191,639 is a continuation-in-part of application No. 09/415,382, filed on Oct. 8, 1999, now Pat. No. 6,419,704.
Application No. 10/191,639 is a continuation-in-part of application No. 09/580,231, filed on May 26, 2000, now Pat. No. 6,494,883.
(60) Provisional application No. 60/488,048, filed on Jul. 17, 2003.

(51) Int. Cl. A61B 17/56

(52) U.S. Cl. 606/61

(57) ABSTRACT

Intradiscal components associated with Total Disc Replacements (TDRs), for example, are maintained in a disc space with keels having attributes that resist extrusion, pull-out, and/or backout. In the preferred embodiment, the keel is curved to resist extrusion, particularly anterior or posterior extrusion. The invention may include a TDR with a pair of endplates, each with a keel extending into a different vertebral body, and wherein the keels are angled or curved in different directions to resist extrusion. In alternative embodiments, the keel may include one or more members that extend outwardly to resist extrusion. Such members may be spring-biased, composed of a shape-memory material, or extend outwardly in response to an applied mechanical force, as might be applied by turning a screw. The keel may further include a bone-ingrowth plug or coating or ‘teeth’ to resist extrusion. Keels according to the invention may also be configured to resist extrusion through the addition of an elongate member that penetrates a vertebral body and the keel. Such a member may be a secondary keel or screw.

### REFERENCE TO RELATED APPLICATION

This application claims priority from U.S. Provisional Patent Application Ser. No. 60/488,048, filed Jul. 17, 2003; and is a continuation-in-part of U.S. patent application Ser. No. 10/007,477, filed Nov. 8, 2001, which is a continuation-in-part of U.S. patent application Ser. No. 09/483,805, filed Jan. 15, 2000, now U.S. Pat. No. 6,432,107.

This application is also a continuation-in-part of U.S. patent application Ser. No. 10/652,842, filed Aug. 29, 2003.

This application is also a continuation-in-part of U.S. patent application Ser. No. 10/303,385, filed Nov. 25, 2002, which is a continuation-in-part of U.S. patent application Ser. No. 10/191,639, filed Jul. 9, 2002, which is a continuation-in-part of U.S. patent application Ser. No. 09/415,382, filed Oct. 8, 1999, now U.S. Pat. Nos. 6,419,704, and 09/580,231, filed May 26, 2000, now U.S. Pat. No. 6,494,883.

This application is also a continuation-in-part of U.S. patent application Ser. No. 10/______, filed Jul. 16, 2004.

The entire content of each application and patent are incorporated herein by reference.

### FIELD OF THE INVENTION

This invention relates generally to artificial disc replacements (ADRs) and, in particular, to ADRs with extrusion-resistant keel configurations.

### BACKGROUND OF THE INVENTION

Prior-art artificial disc replacements (ADRs) typically include parallel “keels” that penetrate into the vertebral endplates to resist lateral movement. However, such keels do not resist movement of the ADR in other directions, such as posterior-to-anterior directions. Extrusion of ADRs typically occurs anteriorly. Extrusion of a TDR from the disc space can cause a fatal injury to the great vessels.

### SUMMARY OF THE INVENTION

This invention improves upon the existing art by providing intradiscal components such as Total Disc Replacements (TDRs) that are maintained in the disc space with keels having attributes that resist extrusion, pull-out, and/or backout. Such components typically include an endplate adapted to contact the inferior or superior endplate of a vertebral body and an extrusion-resistant keel that extends from the endplate and into a portion of the vertebral body.

In the preferred embodiment, the keel is curved to resist extrusion, particularly anterior or posterior extrusion. The invention may include a TDR with a pair of endplates, each with a keel extending into a different vertebral body, and wherein the keels are angled or curved in different directions to resist extrusion. Keels according to the invention may be integral to their respective endplates, or they may separate components, facilitating assembly after the endplate is in contact with the inferior or superior endplate of a vertebral body.

In alternative embodiments, the keel may include one or more members that extend outwardly to resist extrusion. Such members may be spring-biased, composed of a shape-memory material, or extend outwardly in response to an applied mechanical force, as might be applied by turning a screw. The keel may further include a bone-ingrowth plug or coating or ‘teeth’ to resist extrusion.

Keels according to the invention may diverge or converge to resist anterior or posterior ADR extrusion. Keels according to the invention may also be configured to resist extrusion through the addition of an elongate member that penetrates a vertebral body and the keel. Such a member may be a secondary keel or screw. The keel may be placed in a non-central location to facilitate revision.

### BRIEF DESCRIPTION OF THE DRAWINGS

FIG. 1A is a top view of a superior ADR endplate (EP) of the present invention;

FIG. 1B is a bottom view of an inferior ADR EP with the keel curving in an opposite direction;

FIG. 1C is a view of the front of the ADR drawn in FIGS. 1A and 1B;

FIG. 1D is a view of the front of the spine and a slot-cutting guide;

FIG. 1E shows a curved slot-cutting tool;

FIG. 2 is a view of the front of an alternative ADR with one or more keels placed in a non-central location;

FIG. 3 is a view of the front of an ADR with a keel (or keels) limited to one of the ADR EPs;

FIG. 4 is a view of the front of an ADR with keels on the top and bottom;

FIG. 5 is a front view of an ADR embodiment wherein the keels are added after the ADR is placed into the disc space;

FIG. 6A is a view of the side of a TDR with keels incorporating an anti-extrusion feature according to the present invention;

FIG. 6B is a view of the top of the TDR drawn in FIG. 6A;

FIG. 6C is a view of the top of the TDR drawn in FIG. 6A;

FIG. 7 is the view of the side of an alternative embodiment;

FIG. 8A is a view of the front of a TDR with an alternative embodiment;

FIG. 8B is a view of the front of the TDR drawn in FIG. 8A;

FIG. 8C is a view of the side of the TDR drawn in FIG. 8A;

FIG. 8D is a sagittal cross section of the TDR drawn in FIG. 8A;

FIG. 9A is a view of the side of a TDR with an alternative embodiment with keels that contain a screw and a screw-activated expanding member;

FIG. 9B is an axial cross section of the TDR drawn in FIG. 9A;

FIG. 9C is a view of the top of the TDR drawn in FIG. 9A;

FIG. 10A is a view of the side of a TDR with an alternative embodiment;

FIG. 10B is a view of the top of a TDR with an alternative embodiment of the keels;

FIG. 11A is a view of the top of a TDR with an alternative embodiment of the present invention;

FIG. 11B is a view of the top of a TDR with an alternative embodiment;

FIG. 11C is a view of the top of the TDR drawn in FIG. 11A with a removable drill guide and a drill;

FIG. 12A is a view of the top of a TDR with an alternative embodiment;

FIG. 12B is a view of the top of the TDR drawn in FIG. 12A;

FIG. 13 is a view of the front of a TDR with an embodiment of the keels;

FIG. 14A is an axial cross section of a TDR with an embodiment of the keels; and

FIG. 14B is an axial cross section of the TDR drawn in FIG. 14A.

### DETAILED DESCRIPTION OF THE INVENTION

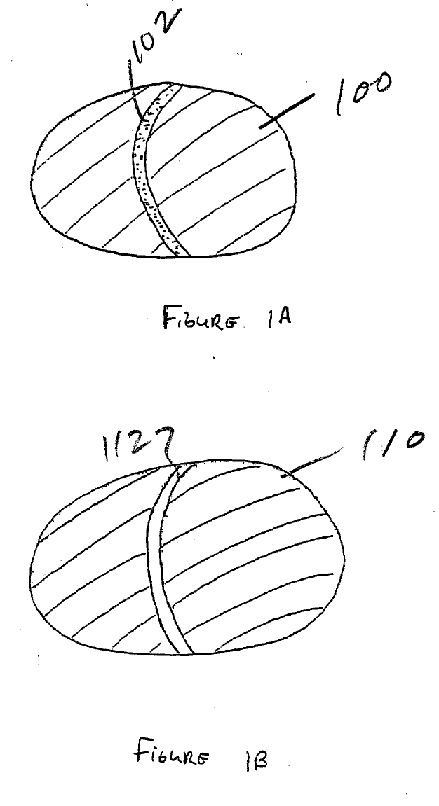

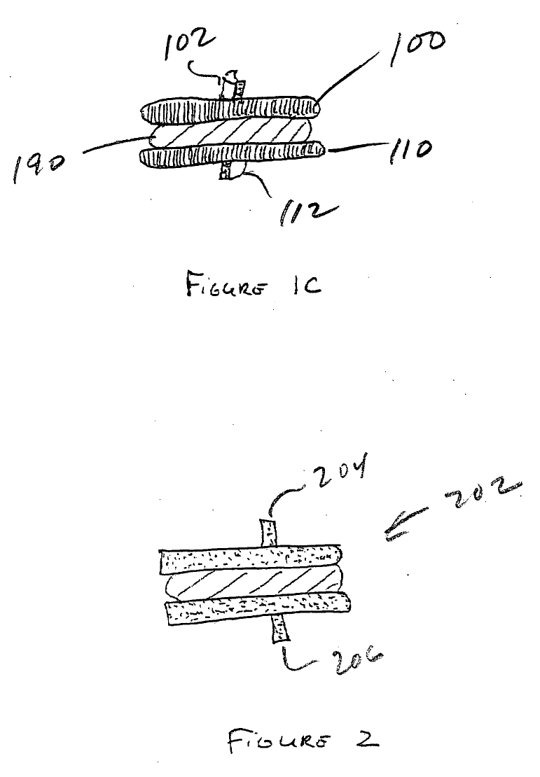

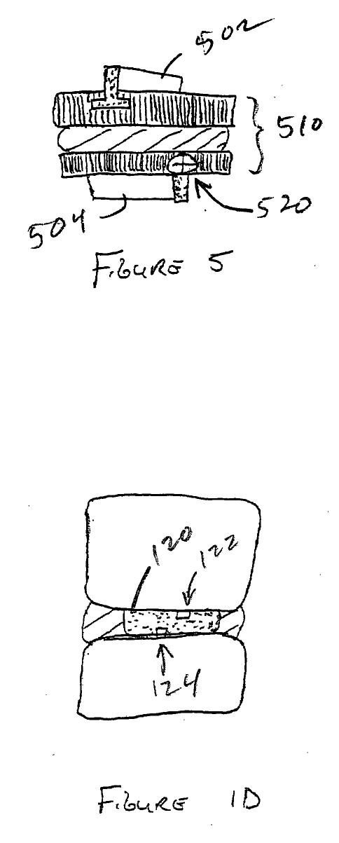

FIG. 1A is a top view of a superior ADR endplate (EP) 100 with a curved keel 102 according to the invention. The keel 102 is rotated into a corresponding slot formed in a vertebral body. Like a screw, ADRs with curved keels would need to see rotational forces to extrude. Since ADRs experience minimal rotational forces, extrusion is unlikely. FIG. 1B is a bottom view of an inferior ADR EP 110 with the keel 112 curving in an opposite direction. Thus, the ADR EPs must see rotational forces in opposite directions to extrude. FIG. 1C is a view of the front of the ADR drawn in FIGS. 1A and 1B. Spacer 190 can be of any suitable configuration, including the use of one or more rigid articulating components, compressible materials, and so forth; the embodiments disclosed herein are not limited in this regard.

FIG. 1D is a view of the front of the spine and a slot-cutting guide 120. The cutting guide has slots 122, 124 to direct a curved slot-cutting tool 130 shown in FIG. 1E to cut curved slots into the vertebrae. The tool preferably includes transverse component 132 that impinges against the slot-cutting guide to prevent the cutting tool from entering the spinal canal. The position of the guide is preferably confirmed with radiologic imaging prior to cutting slots for the keels. A tool (not shown) may be reversibly attached to the slot-cutting guide to hold the guide in position while cutting slots in the vertebrae.

FIG. 2 is a view of the front of an alternative ADR 202 with one or more keels 204, 206 placed in a non-central location. Keels that are located in non-central locations can be used to revise ADRs with centrally located keels. The non-central keels can be placed into new slots created in the vertebrae following ADR removal. The non-central keels can also be used to avoid important vascular structures. Wide, centrally located keels (wider than 3 mm) could also be used when revising ADRs with thin (less than 3 mm) keels.

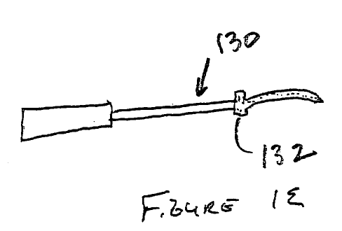

FIG. 3 is a view of the front of an ADR with a keel 302 (or keels) limited to one of the ADR EPs 304. This embodiment avoids cutting slots into the top and bottom of an intermediate vertebra between ADRs at adjacent levels. The top ADR EP of the top ADR would have a keel, while the bottom of the bottom ADR would also have a keel (or vice-versa)

FIG. 4 is a view of the front of an ADR with keels 402, 404 on the top and bottom ADR EPs 406, 408 that are oriented in different directions.

FIG. 5 is a front view of an ADR embodiment wherein the keels 502, 504 (or a single keel) are added after the ADR 510 is placed into the disc space. The keels are reversibly attached to the ADR. For example, the keels could fit into grooves in the ADR EPs. A screw such as 520 could be used to hold the keels in position.

The keels according to this invention may be metallic, ceramic, or composed of a bioresorbable material. Applicable resorbable polymers include the alphapolyesters such as polyactic acid (PLA) and polyglycolic acid (PGA). A combination of PLA and PGA could be used. Other polymers could be used including: poly(ortho esters), poly(glycolide-cotrimethylene carbonate), polyanhydrides, poly-n-dioxanone), poly(a-caprolactone), poly-L-lactide-co-6-caprolactone, poly(a-hydroxybutyrate), poly(a-hydroxyvaleric acid), and pseudopoly(amino acids). Resorbable keels would facilitate revision surgery. The sides of the keels could be covered with a rough plasma spray of bone-ingrowth promoting material, such as Titanium. The friction between the sides of the keels and the plasma spray would also help prevent extrusion of the TDR.

Additional methods and apparatus for ADR extraction are taught in my co-pending application U.S. Ser. No. 10/741,290. My co-pending application U.S. Ser. Nos. 10/657,914 and 10/607,652 teach the use of resorbable materials to temporarily limit ADR motion. The novel resorbable ADR fixation components taught in this application do not restrict movement across the articulating surfaces of the ADR. Any ADR fixation component, including the embodiments of the fixation components taught in this application (keels, screws, protruding components, and expanding components could be made of resorbable material. The plate-like components for ADRs as taught in my co-pending application U.S. Ser. No. 60/538,179 could also be made of resorbable materials.

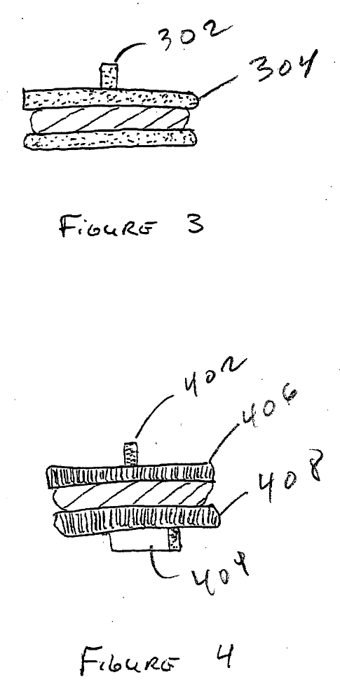

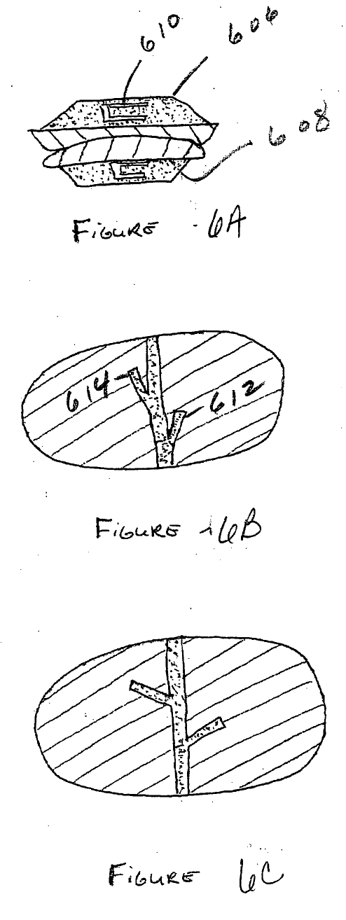

FIG. 6A is a view of the side of a TDR with keels 606, 608 incorporating an anti-extrusion feature according to the invention. Each keel has at least one spring-biased portion 610. FIG. 6B is a view of the top of the TDR drawn in FIG. 6A. Spring biased portions 612, 614 are in a first, compressed position. FIG. 6C is a view of the top of the TDR drawn in FIG. 6A. The spring-biased components are in a second, extended position. The TDR is inserted in a direction that deflects the spring-biased components. The TDR drawn in FIG. 6C would be inserted in the direction from the bottom of the TDR to the top of the TDR. Once extended, the spring-biased components resist movement of the TDR in the direction from which it was inserted. The extending components could also be made of shape-memory material. This embodiment of the invention is similar to that drawn in FIG. 4B of U.S. Pat. No. 6,432,107.

FIG. 7 is the view of the side of an alternative embodiment wherein holes 702 within the keels can be filled with bone, an osteoconductive material, or an osteoinductive material including BMP. Alternatively, cement could be injected through a port in the front of the keels. The cement could fill the holes in the keels. The cement would not cover the tops and bottoms of the TDRs. Thus, the top and bottom surfaces of the TDR would be available for bone ingrowth.

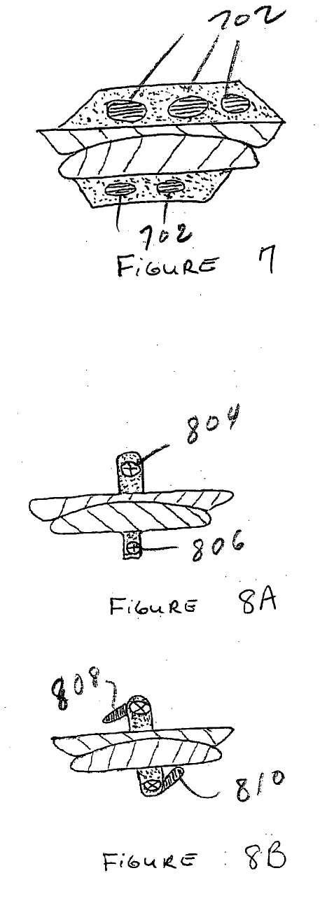

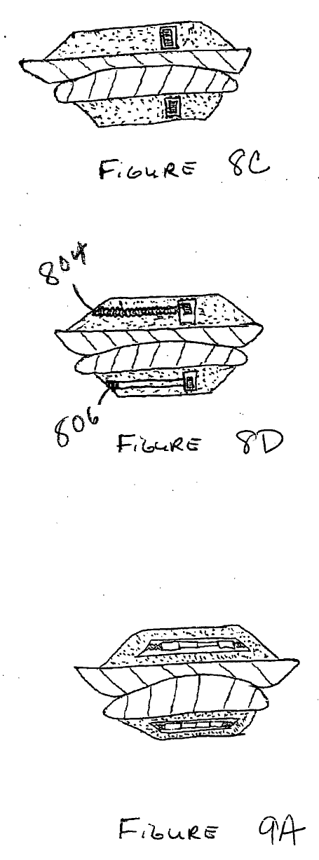

FIG. 8A is a view of the front of a TDR with an alternative embodiment wherein screws 804, 806 in the front of the keels deploy rotating members. FIG. 8B is a view of the front of the TDR drawn in FIG. 8A. The anti-extrusion members 808, 810 have been rotated into position. FIG. 8C is a view of the side of the TDR drawn in FIG. 8A. FIG. 8D is a sagittal cross section of the TDR drawn in FIG. 8A. The screws used to rotate the anti-extrusion members are illustrated at 804, 806.

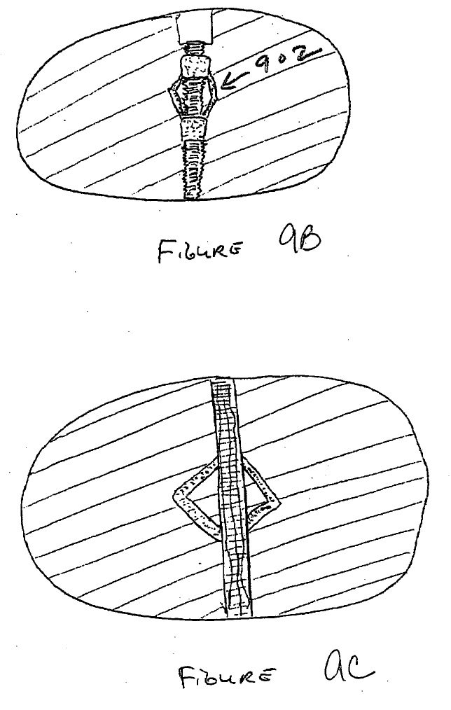

FIG. 9A is a view of the side of a TDR with an alternative embodiment with keels that contain a screw and a screw-activated expanding member. FIG. 9B is an axial cross section of the TDR drawn in FIG. 9A. The expanding member 902 is drawn in its first, compressed position. FIG. 9C is a view of the top of the TDR drawn in FIG. 9A. The expanding member 904 is seen in its, second, expanded position.

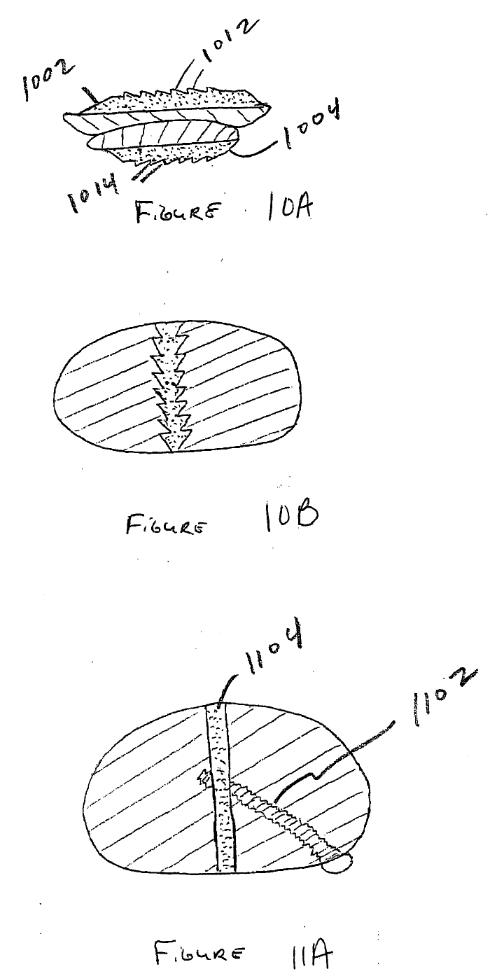

FIG. 10A is a view of the side of a TDR with an alternative embodiment wherein the top of the keels 1002, 1004 have teeth 1012, 1014 that resist extrusion of the device. The teeth may be slanted to assist insertion of the TDR. The TDR drawn in FIG. 10A would be easiest to insert from the right side of TDR leading the insertion. FIG. 10B is a view of the top of a TDR with an alternative embodiment of the keels. The sides and/or the top of the keels may have teeth similar to those drawn in FIG. 10A.

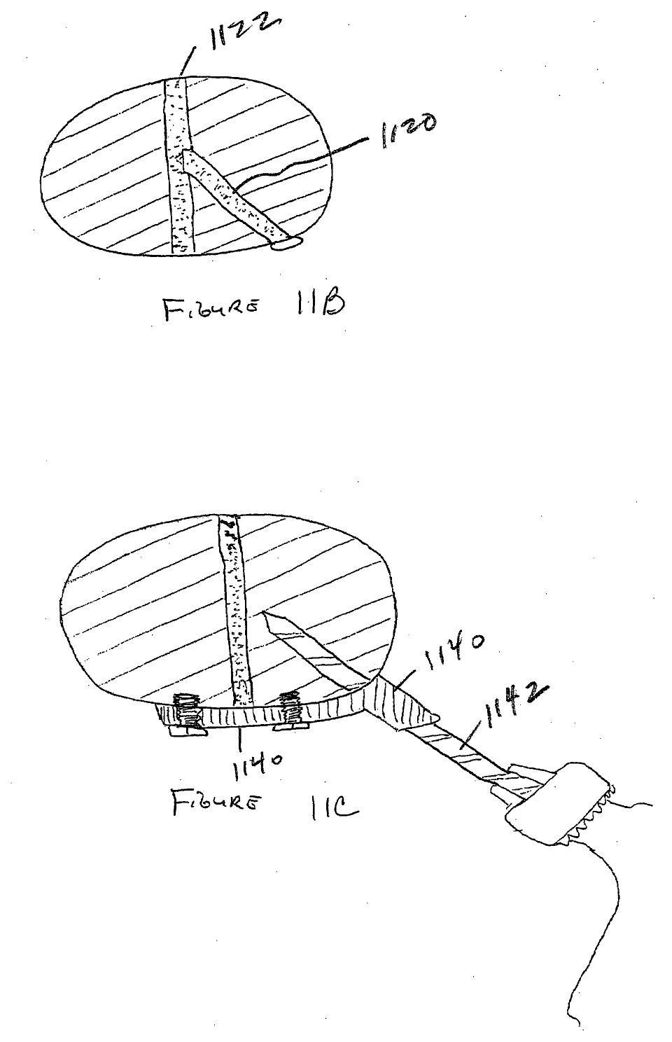

FIG. 11A is a view of the top of a TDR with an alternative embodiment of the invention. A guide similar to that drawn in FIG. 6C of co-pending U.S. patent application Ser. No. 10/007,477, is used to direct a screw 1102 into the keel 1104. The screw can extend through the opposite side of the vertebra. Alternatively, the screw may thread and lock into the keel without extending through the keel. The screw or the keel could be made of a bioresorbable material.

FIG. 11B is a view of the top of a TDR with an alternative embodiment wherein a second keel component 1120 is connected to a first keel component 1122. The second keel component could be held in place by a screw through the center of the second keel. FIG. 11C is a view of the top of the TDR drawn in FIG. 11A with a removable drill guide 1140 and a drill 1142. In the preferred embodiment, the screw or second keel component is added to the left side of a keel of a TDR that is inserted from an anterior approach to the spine; the screw or second keel component is preferably added to the front of a keel of a TDR that is inserted through a lateral approach to the spine.

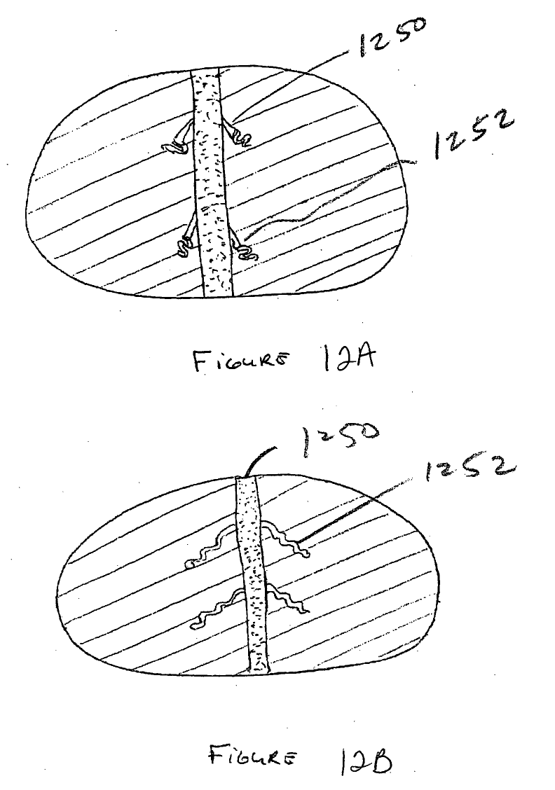

FIG. 12A is a view of the top of a TDR with an alternative embodiment wherein the keels contain components 1250, 1252 made of a shape-memory material, such as Nitinol. The shape-memory components lengthen after insertion of the TDR into the disc space. FIG. 12B is a view of the top of the TDR drawn in FIG. 12A. The shape memory components are drawn in their second, lengthened, position.

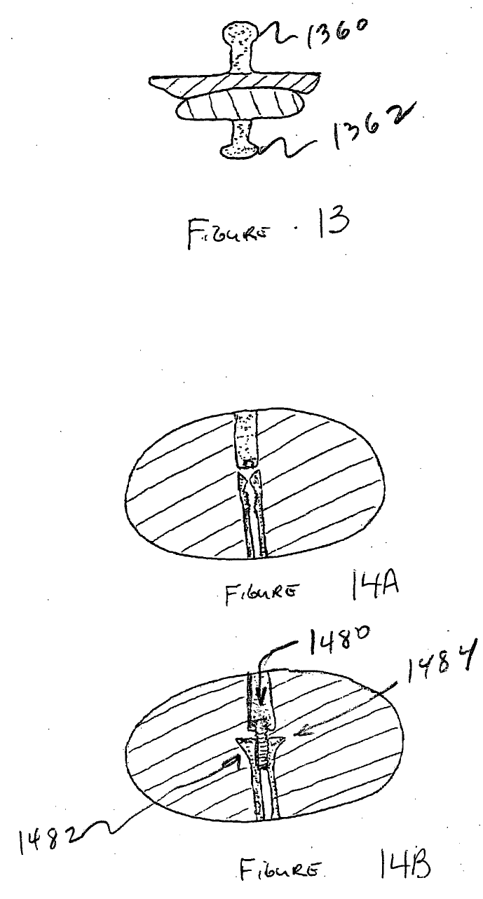

FIG. 13 is a view of the front of a TDR with an embodiment of the keels 1360, 1362 similar to those depicted in FIG. 16A of U.S. patent application Ser. No. ______. The keels are shaped to prevent the keels from pulling out of the vertebrae. The enlargements at the top and bottom of the keels cooperate with slots milled into the vertebrae.

FIG. 14A is an axial cross section of a TDR with an embodiment of the keels similar to that drawn in FIG. 21A of U.S. patent application Ser. No. ______. FIG. 14B is an axial cross section of the TDR drawn in FIG. 14A. A screw 1480 has been inserted to force the walls of the keel into an extended position.

I claim:

1. An intradiscal device, comprising:
an endplate adapted to contact the inferior or superior endplate of a vertebral body;
a keel extend from the endplate and into a portion of the vertebral body; and
wherein the keel is configured to resist extrusion.

2. The intradiscal device of claim 1, wherein the keel is curved.

3. The intradiscal device of claim 1, including a pair of endplates, each with a keel extending into a different vertebral body; and
wherein the keels are angled or curved in different directions to resist extrusion.

4. The intradiscal device of claim 1, wherein the keel is a separate component which is added to the endplate.

5. The intradiscal device of claim 4, wherein the keel is added after the endplate is in contact with the inferior or superior endplate of a vertebral body.

6. The intradiscal device of claim 1, wherein the keel includes one or more members that extend outwardly to resist extrusion.

7. The intradiscal device of claim 6, wherein the members are spring-biased to extend outwardly.

8. The intradiscal device of claim 6, wherein the members are composed of a shape-memory material to extend outwardly.

9. The intradiscal device of claim 6, wherein the members extend outwardly in response to an applied mechanical force.

10. The intradiscal device of claim 9, wherein the mechanical force is applied by turning a screw.

11. The intradiscal device of claim 1, wherein the keel includes teeth to resist extrusion.

12. The intradiscal device of claim 1, wherein the keel includes a bone-ingrowth plug or coating to resist extrusion.

13. The intradiscal device of claim 1, wherein the keel is configured to resist extrusion through the addition of an elongate member that penetrates a vertebral body and the keel.

14. The intradiscal device of claim 1, including a keel placed in a non-central location to facilitate revision.

15. The intradiscal device of claim 1, including a plurality of keels that diverge or converge.
